(19) United States
(12) Patent Application Publication
(10) Pub. No.: US 2010/0204704 A1
(43) Pub. Date: Aug. 12, 2010

(54) Device for Inserting an Intraocular Lens into an Eye

(75) Inventors: Nathaniel Davies, Buckinghamshire (GB); Michael Ring, Buckinghamshire (GB); Peter Toop, Buckinghamshire (GB)

Correspondence Address: ANDRUS, SCEALES, STARKE & SAWALL, LLP, 100 EAST WISCONSIN AVENUE, SUITE 1100, MILWAUKEE, WI, 53202, US

(73) Assignee: Rayner Intraocular Lenses Ltd., Chesham, Buckinghamshire (GB)

(21) Appl. No.: 12/669,436

(22) PCT Filed: Jul. 16, 2008
(86) PCT No.: PCT/GB08/02438

§ 371 Date: Apr. 26, 2010

Related U.S. Application Data
(60) Provisional application No. 60/951,312, filed on Jul. 23, 2007.

(30) Foreign Application Priority Data
Jul. 18, 2007 (GB) 0714005.6

(51) Int. Cl. A61F 9/007

(52) U.S. Cl. 606/107

(57) ABSTRACT

The present invention provides accurate, low cost devices that have application in the field of intraocular lens delivery. Specifically, the present invention provides a device for inserting an intraocular lens into an eye, comprising a body having a lumen which is capable of accommodating an intraocular lens and a rod which is capable of pushing said intraocular lens out of the lumen and into the eye, wherein the rod has an intraocular lens-contacting tip which has a first configuration having a larger radial cross-sectional profile than a second configuration thereof. The invention further provides a device for inserting an intraocular lens into an eye, comprising a body having a lumen which is capable of accommodating an intraocular lens and a rod which is capable of pushing said intraocular lens out of the lumen and into the eye, wherein body lumen has a guide which is adapted to engage with a projection on the rod, such that the tip of the rod is capable of being maintained at a predetermined distance from the wall of the body lumen as it is moved along at least a portion thereof.

### TECHNICAL FIELD

The present invention relates to an intraocular lens injector for injecting an intraocular lens into an eye of a patient.

### BACKGROUND OF THE INVENTION

One of the operative treatments used to treat cataract is a method of removing a crystalline lens from an eye of a patient and then injecting an intraocular lens in place of the crystalline lens. To inject the intraocular lens, the following steps are usually performed: first making an incision in the eye; fragmenting and aspirating a clouded crystalline lens through the incision; and then injecting the intraocular lens into the eye through the incision to implant it in place of the crystalline lens.

If a large incision is made, it may become a burden on the eye and also cause astigmatism of the eye after the operation. To prevent such disadvantages, an intraocular lens injector is used to inject a foldable intraocular lens into an eye through a smaller incision. In this injector, the foldable intraocular lens held in a housing of the injector is pushed toward the tip of the injector while being folded into a smaller shape. Thereafter, the folded intraocular lens is pushed out of the tip of the injector inserted in the eye through the incision and is spread (unfolded) and placed in the eye.

### SUMMARY OF THE INVENTION

The present invention provides accurate, low cost devices that have application in the field of intraocular lenses and their delivery.

Thus, in a first aspect, the present invention provides a device for inserting an intraocular lens into an eye, comprising a body having a lumen which is capable of accommodating an intraocular lens and a rod which is capable of pushing said intraocular lens out of the lumen and into the eye, characterised in that the rod has an intraocular lens-contacting tip which has a first configuration having a larger radial cross-sectional profile than a second configuration thereof.

In a second aspect, the present invention provides a device for inserting an intraocular lens into an eye, comprising a body having a lumen which is capable of accommodating an intraocular lens and a rod which is capable of pushing said intraocular lens out of the lumen and into the eye, characterised in that body lumen has a guide which is adapted to engage with a projection on the rod, such that the tip of the rod is capable of being maintained at a predetermined distance from the wall of the body lumen as it is moved along at least a portion thereof.

As used herein, the term “profile” means the area of the end face of the rod tip which contacts the intraocular lens and/or the area of a radial cross-section taken though the end portion of the rod tip perpendicular to the longitudinal axis of the rod. Preferably, the term “end portion” means the final ⅓, more preferably the final ¼, more preferably the final ⅕ of the length of the rod.

Preferably, the rod is 50-250 mm long, more preferably 75-200 mm long, more preferably 100-175 mm long. Preferably, the “end portion” of the rod is the final 35 mm or less of the end of the rod which, in use, contacts the intraocular lens, more preferably, the final 25 mm, most preferably, the final 10 mm.

Prior art intraocular lens delivery devices have suffered from problems of blockage. This may be caused by the radial deflection of a rod, in particular, the tip of the rod where it comes into contact with the intraocular lens, within the lumen of the device. This may occur because the rod is made from material (such as plastic) which deflects under its own weight within the lumen of the device. Alternatively or additionally, the rod may undergo radial deflection within the lumen of the device when it comes into contact with, and is pushed against the intraocular lens. The radial deflection of the tip of the rod may cause the intraocular lens to become trapped between the tip of the rod and the wall of the lumen, thereby preventing the lens from being ejected from the device. Furthermore, the intraocular lens may become damaged when it is trapped between the rod tip and the lumen wall. The present invention solves these and other problems by controlling the position of the rod tip as it moves along the body lumen. For example, the device is capable of substantially preventing or restraining the radial displacement of the rod tip within the body lumen, at least until it comes into contact or close proximity with the intraocular lens. This allows the contact between the rod tip and the intraocular lens to be controlled. The radial displacement of the end portion of the rod may be controlled throughout the entire longitudinal transition of the rod down the body lumen.

Alternatively or additionally, the rod tip may enter into the fold of the intraocular lens, rather than pushing the edge of the lens or its haptics. This can lead to bunching of the lens against the body lumen wall or the injector tip. The present invention solves this problem by controlling the position of a rod tip. This is particularly useful where the rod tip has a smaller cross-sectional profile than the body lumen. The present invention enables the rod tip to be controlled such that it contacts the intraocular lens in a predetermined position so as to reduce or eliminate the likelihood of entering the folded intraocular lens.

As used herein, the term “close proximity” means less than 10 mm, more preferably, less than 5 mm.

Preferably, when in the second configuration, the lens contacting tip of the rod is radially displaced less than 1 mm, more preferably, less than 0.5 mm, more preferably less than 0.1 mm from the longitudinal axis of the rod. Most preferably, there is substantially no radial displacement of the rod from its longitudinal axis.

Preferably, there is a stepped transition between the first and second configurations of the rod tip. For example, preferably, the rod tip in the first configuration has a first cross-sectional profile which switches between cross-sectional profile of the first configuration and the second configuration at a discrete point. Preferably the transition between the first and second configuration is not effected by compression of the rod tip i.e., not in a continuous manner as a function of the longitudinal displacement of the rod within the body lumen and/or injector tip. Preferably, the rod has only two tip configurations, having a distinct stepped transition between the two configurations.

Preferably, the cross-sectional profile of the second configuration of the rod tip is 5-95% smaller than the first configuration, more preferably 10-75% smaller, more preferably 25-50% smaller.

The injector device of the present invention preferably comprises an injector tip through which the intraocular lens may be expelled from the device. The injector tip is preferably substantially co-axial with the body lumen of the device. The injector tip is preferably substantially axially co-linear with the body lumen of the device. The injector tip may be formed integrally with, or separately to the body portion of the device. This injector tip is preferably capable of being inserted through an incision in the eye such that the intraocular lens may be delivered to a location within the eye.

The injector tip preferably comprises a lumen having a radial cross-sectional area which is on average smaller than the average radial cross-sectional area of the body lumen. Preferably, the lumen of the injector tip has a tapering radial cross-sectional area which decreases towards the injector tip (i.e., the tip through which the intraocular lens is delivered into the eye).

The injector tip preferably comprises an open mouth with an outside diameter of no more than 3.0 mm, more preferably no more than 2.0, more preferably no more than 1.8 mm, preferably about 1.4 mm.

The injector tip may have portions that are symmetrically or asymmetrically tapered.

The injector tip is preferably tapered towards its open end in order to present a smaller radial cross-sectional area at the tip. This reduces the trauma caused when the injector tip is inserted into the eye. However, as a consequence of the decreasing cross-sectional profile of the injector tip, a rod having larger radial cross-sectional profile would not be able to push the intraocular lens all the way to the tip thereof. This is because the reduced cross-section of the tip lumen would restrict the translational movement of the rod therein. One solution to this problem may be to provide a rod having a radial cross-sectional profile which matches that of the injector tip of the device. However, this means that the cross-sectional profile of the end of the rod will be smaller than the body lumen. This enables the possibility that the rod may become radially displaced within the body lumen, thereby leading to the problems described above. The present invention provides a solution to these problems by providing a rod having a tip which contacts the intraocular lens, the tip having a first cross-sectional profile which approximates the cross-sectional profile of the body lumen. Thus, the radial displacement of the rod may be controlled throughout the entire length of its movement within both the body lumen and the injector tip of the device, i.e. in both configurations of the tip of the rod.

The shape of the end face of the rod tip and/or the radial cross-section taken though the end portion of the rod tip preferably have substantially the same shape, although not necessarily the same area, as the body lumen and/or the tip lumen.

Preferably, the profile of the rod tip is approximately circular in cross-section. Preferably, the radial cross-sections of the body lumen and the tip lumen are approximately circular.

The rod tip may be provided with a depression or may be otherwise profiled to accommodate and/or manipulate at least a portion of the intraocular lens.

The rod preferably comprises two parts which are adapted for longitudinal movement relative to one another. Preferably, the relative longitudinal movement is effected within the body lumen and/or the injector tip of the device. Preferably, the two parts are adapted for controlled relative longitudinal movement between two end points. Preferably the relative longitudinal movement of the two rod parts enables the transition between the first and second configurations of the rod.

The rod preferably comprises at least two parts, preferably separate parts, which are adapted to form, in use, a single rod. Preferably, at least a portion of the rod is complimentary to the shape of the body lumen, thereby enabling longitudinal movement along at least a portion thereof, and preferably the whole length thereof.

The body lumen may extend a portion or the whole length of the body of the device. For example, the body lumen may comprise 20-100% of the length of the body of the device, more preferably, 25-90%, more preferably, 30-75% of the length of the body of the device.

The rod preferably comprises at least two parts which, in the first configuration, have a cross-sectional profile complimentary to that of the body lumen. The rod is capable of relative longitudinal movement within the body lumen. At a pre-determined point of longitudinal displacement towards the injector tip, at least a first part of the rod is capable of being selectively detached from one or more other parts of the rod, thereby enabling relative longitudinal movement of the first part of the rod to the other part(s) of the rod. Thus, the one or more other parts of the rod are able to continue their longitudinal movement along the body lumen towards the injector tip while the first part substantially remains in the pre-determined position within the body lumen and/or the injector tip. This constitutes the transition between the first and the second configuration of the rod. The longitudinal position of the first rod part may be maintained relative to the other parts by the inclusion of restraining and/or attachment means. For example, a détente, catch or the like may be included on one or more of the rod parts. This may be adapted to engage one or more other constituent parts of the rod, for example, a notch or depression formed therein. Alternatively or additionally, one or more of the rod parts may be provided with a surface which offers frictional resistance with its interface with one or more other constituent parts of the rod. Such frictional resistance may be provided by patterning or etching of the parts.

Where a restraining and/or attachment means is present (e.g., a détente), this is capable of being detached from the one or more other rod parts at a predetermined point, thereby enabling the other parts to continue their longitudinal movement down the body lumen towards the injector tip. The “predetermined point” of detachment may be one that is automatically actuated under normal longitudinal movement of the rod within the body lumen. Alternatively, the “predetermined point” of detachment may be one that the user has to actuate separately from merely pushing the rod along the body lumen.

Where frictional resistance is used to restrict the relative movement of the rod parts, the rod parts are preferably adapted in order that the frictional resistance may be overcome when the rod has travelled a predetermined longitudinal distance within the body lumen and meets a stop, partial stop, a decreasing cross-sectional profile of the lumen or a mixture of these. For example, a “stop” may be provided by a flange within the body lumen. The same means may be used to effect the detachment of restraining and/or attachment means from one or more rod parts.

In one embodiment, one of the rod parts is resiliently biased transverse to the longitudinal axis of the rod. In this embodiment, the rod part is preferably hinged, scored, cast, moulded or a mixture thereof in order to provide such resilient bias. At the pre-determined point of longitudinal displacement of the rod within the body lumen, at least a portion of this rod part can become detached from the one or more other rod parts by virtue of its resilient bias. Preferably, this resiliently biased portion of rod contains the restraining and/or attachment means. In this respect, the body lumen may be provided with means for accommodating the radial displacement of the resiliently biased portion away from the other part(s) of the rod. For example, the body lumen may be provided with a chamber, recess, relatively large diameter (relative to the rest of the body lumen) or the like, which is capable of accommodating such radial displacement.

Preferably, the rod comprises two parts which form a substantially cylindrical cross-section which is substantially complimentary to the cross-sectional profile of the body lumen in the first configuration. Upon transition to the second configuration, the cross-sectional profile of the rod tip is preferably substantially complimentary to that of the narrowest part of the injector tip.

In a preferred embodiment, the rod comprises two parts having substantially semi-circular prismatic portions. These may be assembled to form a rod having at least a portion which is a substantially cylindrical prism. At least a portion of this cylindrical prism, preferably its whole, is adapted to be substantially complimentary to at least a portion of the body lumen, preferably the whole of the body lumen. Preferably, one of the rod parts is adapted to engage with the other rod part(s). For example, one of the rod parts may comprise a guide which is capable of engaging with a complimentary portion(s) of another rod part. This enables the movement of the constituent rod parts to be controlled relative to one another and, preferably, relative to the body lumen. For example, such a configuration may be used to control the rotation of the rod within the body lumen/injector tip.

The body lumen and/or the injector tip may comprise a guide which can be used to control the rotation of the rod within the device. The guide may be axially co-linear with the body lumen. Alternatively, the guide may be formed in a helical or curved shape, thereby imparting a rotation on the rod as it moves relative to the body lumen.

In one embodiment, the rod may comprise at least two parts having a telescoping action, thereby enabling the transition between the first and second configuration. For example, the rod may comprise a main, substantially cylindrical portion having one or more co-axial sleeves or collars. The main portion of the rod may comprise one or more neck portions. The neck portion(s) may be adapted to accommodate and/or retain the sleeve or collar thereon. Restraining and/or attachment means may be formed in or attached to either one or more sleeves or collars, or to the main portion of the rod. Complimentary parts to the restraining and/or attachment means may be formed on the part(s) not containing the restraining and/or attachment means. For example, a sleeve or collar part may comprise a détente, and the neck portion may comprise a depression adapted to engage the détente. Preferably, the main portion of the rod is adapted to enable controlled longitudinal movement of the sleeve or collar thereon. Preferably, this longitudinal movement is effected between two end points which preferably define the first and second configurations of the rod respectively.

Preferably, restraining and/or attachment means are adapted such that a relatively small amount of force is required to displace them from their points of engagement. This may be effected by having restraining and/or attachment means with relatively small profiles. Alternatively or additionally, the points of engagement for the restraining and/or attachment means may be adapted to be analogously adapted. For example, a depression may be made relatively shallow, thereby requiring a small force in order to displace a détente or the like therefrom. The restraining and/or attachment means may comprise an annular flange on either the inner surface of the sleeve or the outer surface of the main portion of the rod. This is adapted for engagement with an annular recess in the surface of whichever of the sleeve or rod which does not have the annular flange.

In a further embodiment, the present invention provides a device for inserting an intraocular lens into an eye, comprising a body having a lumen which is capable of accommodating an intraocular lens and a rod which is capable of pushing said intraocular lens out of the lumen and into the eye, characterised in that the rod has an intraocular lens-contacting end portion which is radially collapsible or partially radially collapsible. In this embodiment, the end portion of the rod which is proximate the lens in use may be provided with one or more means for altering its cross-sectional diameter. For example, the rod may comprise a hollow tube or have a hollow tubular portion having a non-constant wall thickness along at least a portion of the rod length, optionally the whole length thereof. In this example, the tubular portion may have one or more relatively thin wall section(s) which would allow the thinned section(s) to collapse, fold, bend, concertina or the like in order to reduce the diameter of the lens-contacting tip relative to the portion of the rod distal to the lens-contacting tip. In an alternative example, the tubular portion may have one or more slots cut into it from the lens contacting tip. The slots are preferably equispaced around the end of the rod. The slot(s) may be tapered away from the lens-contacting tip in order that, in the collapsed configuration, the rod tapered towards its lens contacting tip. Preferably, the rod comprises 1, 2, 3 or 4 slots, most preferably 3. In these embodiments, the rod may comprise one or more of the above-mentioned means for radial constriction, which may be the same or different.

The rod may be provided with a portion at one of its ends which is adapted to be contacted by a user. For example, the rod may be provided with a flattened or plate-like section which may be contacted by a users thumb and/or finger(s), such that the rod may be pushed and/or pulled moved along the body lumen.

The rod may be resiliently biased against transitional longitudinal movement within the body lumen. For example, the device may be provided with a spring or resilient material which provides a force against the depression of the rod within the device. Various configurations of a spring or combination of springs can be applied in the present invention. For example, a coiled spring, cantilever spring, or spring arrangement utilizing the resilient property of the spring's structural configuration and/or the elastic property of the material forming the spring.

The lens injecting device according to the present invention may be configured to apply an opposite force to the advancing rod when the intraocular lens is exiting the injector tip of the device to provide controlled release of the intraocular lens into the eye. In a preferred embodiment, the opposite force is applied to the advancing rod only near the end of its stroke when the intraocular lens is about to exit the tip of the injector. This arrangement allows the rod to freely slide relatively unimpeded most of the distance of its stroke yet provide sufficient back pressure at the end of its stroke to prevent sudden rod advancement as the intraocular lens exits the injector tip.

Preferably, the injector device comprises a lens receiving portion, hereinafter referred to as a load chamber. Preferably, the load chamber is located in or on the body of the device. Preferably, the load chamber is located adjacent to the injector tip of the device. Preferably, the load chamber enables easy insertion of an intraocular lens into the device. Preferably, the load chamber enables the intraocular lens to be folded and/or rolled into a configuration suitable for delivery into the eye. Preferably, the load chamber of the device comprises a loading port. Said loading port preferably has a first, open configuration, wherein the intraocular lens may be loaded into the device, and a second, closed configuration, wherein the intraocular lens is loaded within the device. The load chamber may be formed integrally with the device, or may comprise a cartridge which may be loaded into the device.

The load chamber preferably has an open proximal end and a distal end aligned with the body lumen.

The rod may comprise a sleeve or a partial sleeve which can be used to cover at least a portion of the lens when it is loaded into the load chamber. In this embodiment, the rod may be advanced to just behind the load chamber, i.e., the end of the load chamber furthest from the injector tip. The intraocular lens may then be loaded in to the load chamber and folded or rolled ready for delivery to the eye. At this point, the load chamber is preferably open. The sleeve or a partial sleeve may then be advanced over or around at least a portion or the whole of the lens. The load chamber may then be closed. Thus, the sleeve or partial sleeve may be used to protect the lens and/or keep it in an appropriate configuration, while the load chamber is closed. The rod may then be advanced in order to bring the rod tip into contact with the lens. The lens may then be pushed out of the load chamber. The sleeve or partial sleeve may then be withdrawn to its initial position, or the rod advanced relative to the sleeve or partial sleeve. The intraocular lens may then be pushed out of the injector tip in the usual way according to the present invention.

The rod may comprise a soft tip. The soft tip is preferably solid and formed of a material having a minimum ultimate elongation of 400% and an elastic modulus of between 689 kPa and 2137 kPa at an elongation of 100%. The soft tip may have an ultimate elongation of at least 400%, preferably 780% or greater, and is desirably a thermoplastic elastomer. Preferably, the soft tip has a more rigid insert embedded therein which removably couples to a distal end of the rod.

Preferably, the body of the device is substantially light transparent or light translucent. Preferably, the device comprises light transparent and/or light translucent portions to enable the user to see the intraocular lens when loaded into the device. Preferably, the device adapted such that the intraocular lens may be observed from the point of loading until the point of delivery of the lens.

Preferably, the rod is made from an opaque material, such that it can be seen by a user in situ.

Different parts of the device may comprise different coloured materials or patterning. For example, the tip of the rod or a portion thereof may be coloured or patterned in order that its tip may be clearly seen by a user.

The device of the present invention may be constructed from any suitable materials. The constituent parts of the device of the present invention may comprise one or more polymers. For example, a non-limiting list of suitable polymers that can be independently used to form one or more parts of the device of the present invention includes polyacetals, polyamides, polyimides, polyesters, polycarbonates, polysulphones, polyamide-imides, polyamide-esters, polyamide ethers, polycarbonate-esters, polyamide-ethers, polyacrylates; elastomers such as polybutadiene, copolymers of butadiene with one or more other monomers, butadiene-acrylonitrile rubber, styrene-butadiene rubber, polyisoprene, copolymers of isoprene with one or more other monomers, polyphosphazenes, natural rubber, blends of natural and synthetic rubber, polydimethylsiloxane, copolymers containing the diphenylsiloxane unit; polyalkylmethacrylates, polyethylene, polypropylene, polystyrene, polyvinylacetate; polyvinylalcohol, and polyvinylchloride.

In use, the method of delivering an intraocular lens to the eye preferably comprises placing an intraocular lens in the load chamber, inserting the rod into the proximal end of the body lumen, inserting the open mouth of the injector tip through an incision in the eye, and urging the intraocular lens from the load chamber through the tapered internal lumen of the injector tip. The intraocular lens is urged out of the open mouth of the injector tip and into the eye by the rod.

While the foregoing applies to both aspects of the present invention, the following applies particularly to the second aspect of the present invention.

The guide within the body lumen may comprise a rail, recess or the like. The guide may be axially co-linear with the body lumen. Alternatively, the guide may be formed in a helical or curved shape, thereby imparting a rotation on the rod as it moves relative to the body lumen.

The guide may comprise part of or the whole length of the body lumen, for example 25%, 50%, 75% or 100% of its length. There may be more than one guide, for example 2, 3, 4 or more. Where more than one guide is present, they are preferably located at equidistant intervals around the circumference of the body lumen. For example, where two guides are present, they may be located at 180° intervals to one another. Similarly, where four guides are present, they may be arranged at 90° intervals to one another. Preferably, the guide runs up to the junction of the body lumen with the injector tip.

Preferably, the projection comprises a flap or tab. The projection preferably extends radially from at least a portion of the circumference of the rod. Preferably, the projection has a first end which is attached to the rod and a second end which is capable of engaging the guide. Preferably, the point of attachment of the projection of the rod is frangible, weakened and/or hinged, such that it may bend, break or flex, thereby causing the projection to move out of engagement with the guide. In this regard, the rod may comprise a recess adapted to accommodate the projection if its second end is bent or flexed towards the rod. The rod may comprise more than one projection, for example, 2, 3, 4 or more projections.

Where more than one projection is present, they are preferably positioned to engage individual guides. For example, where two projections are present, they may be located at 180° intervals to one another. Similarly, where four projections are present, they may be arranged at 90° intervals to one another.

Preferably, the projection(s) are located towards the end of the rod used to contact the intraocular lens in use. Preferably, the projection(s) are located within the final ⅓, more preferably the final ¼, more preferably the final ⅕ of the length of the rod. Preferably, the projection(s) are located within the final 35 mm or less of the end of the rod which, in use, contacts the intraocular lens, more preferably, the final 25 mm, most preferably, the final 10 mm.

Preferably, the projection is made from a deformable plastics material.

Preferably, the projection comprises a tab having a depression or notch in its second end which is complimentary to the shape of the guide. Thus, as the rod moves along the body lumen, the depression or notch on the rod engages the guide which controls the radial displacement of the rod relative to the wall of the body lumen.

In a preferred embodiment, the guide is one or more rails. Preferably, the height of the rail from the inner wall of the body lumen is between 0.1-2 mm high, more preferably between 0.5-1.5 mm high, more preferably, 0.75-1.25 mm high. The width of the rail may be constant along its length or it may vary in width. The width of the rail may be constant through its height or it may vary in width. For example, the rail may taper towards its free end (the end which engages the projection of the rod). Alternatively, the rail may have a stepped width, such that it is narrower or wider towards its free end.

In the second aspect of the invention, the cross-sectional profile of the rod tip preferably is complimentary to that of the mouth of the injector tip.

The combination of the guide and the projection can maintain the rod tip at a predetermined distance from the wall of the body lumen, or may hold the rod tip against the wall of the body lumen.

In use, the projection is preferably collapsible at a predetermined position of the longitudinal travel of the rod along the guide. The predetermined position is preferably proximate the region where the diameter of the body lumen or injector tip starts to narrow. Thus, at the predetermined position, the projection preferably contacts a stop, partial stop or the narrowing diameter of the body lumen or injector tip, and the projection is bent as the rod is forced down the body lumen/injector tip. The stop or partial stop may be located on the guide or may depend from the body lumen wall or the surface of the injector tip inner wall. This takes the projection out of engagement with the guide while allowing the tip of the rod to continue its movement towards the open end of the injector tip.

In the second aspect of the present invention, the rod preferably has at least two diameters, which may be separated by a distinct step or may be tapered into one another. The cross-sectional profile of the smaller diameter is preferably 5-95% smaller than the larger diameter, more preferably 10-75% smaller, more preferably 25-50% smaller.

### BRIEF DESCRIPTION OF THE DRAWINGS

FIG. 1a shows a cross-sectional illustration of one embodiment of a device according to the first aspect of the invention.

FIG. 1b shows a cross-sectional illustration of one embodiment of a device according to the first aspect of the invention.

FIG. 1c shows a cross-sectional illustration of one embodiment of a device according to the first aspect of the invention.

FIG. 1d shows a cross-sectional illustration of one embodiment of a device according to the first aspect of the invention.

FIG. 2 shows an enlarged side view of the rod tip guide.

FIG. 3a shows a cross-sectional illustration of a second embodiment of a device according to the first aspect of the invention.

FIG. 3b shows a cross-sectional illustration of a second embodiment of a device according to the first aspect of the invention.

FIG. 3c shows a cross-sectional illustration of a second embodiment of a device according to the first aspect of the invention.

FIG. 4a shows a cross-sectional illustration of an embodiment of a device according to the second aspect of the invention.

FIG. 4b shows a cross-sectional illustration of an embodiment of a device according to the second aspect of the invention.

FIG. 5a shows a perspective view of an embodiment of rod according to the first aspect of the invention.

FIG. 5b shows a perspective view of an embodiment of a rod according to the first aspect of the invention.

FIG. 5c shows an end view of a rod perspective view of an embodiment of a rod according to the first aspect of the invention.

### DETAILED DESCRIPTION OF THE INVENTION

General

The term “comprising” encompasses “including” as well as “consisting” e.g. a device “comprising” X may consist exclusively of X or may include something additional e.g. X+Y.

The term “about” in relation to a numerical value x means, for example, x±10%.

The word “substantially” does not exclude “completely”. Where necessary, the word “substantially” may be omitted from the definition of the invention.

Where a particular feature referred to in the following description is accompanied by a reference numeral, it is not necessary that this feature is explicitly illustrated in the Figures. However, where such a feature is not illustrated, the applicant has endeavoured to indicate that the feature is “not shown” in parentheses. Failure to do so should not be considered limiting the scope of invention in any way.

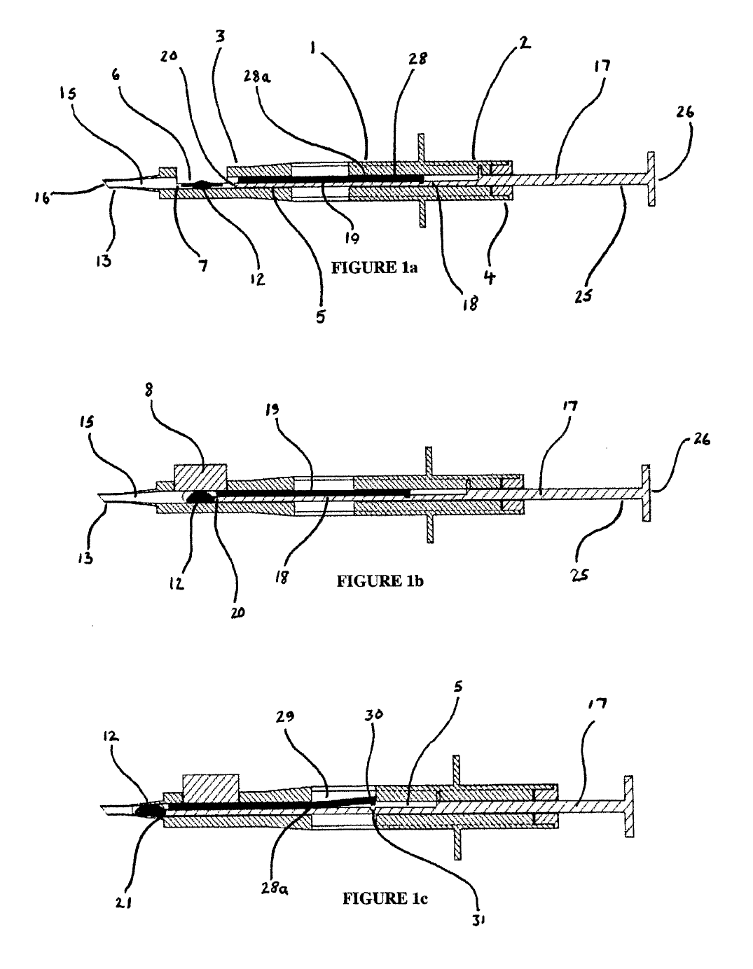

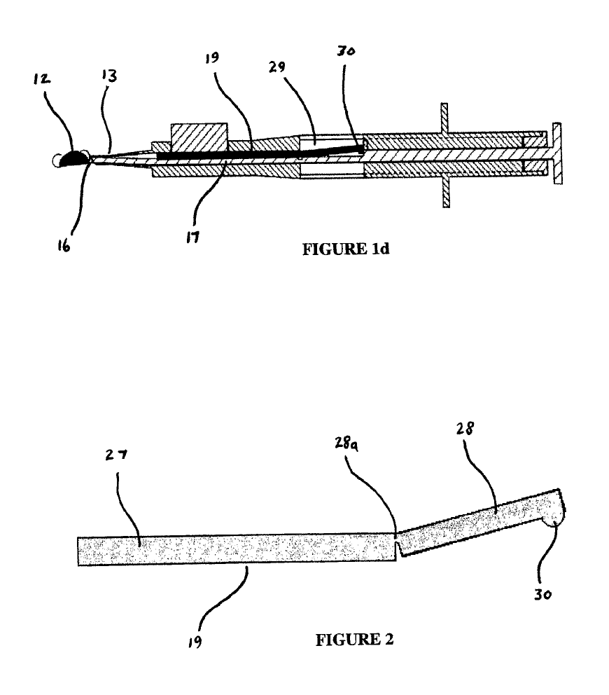

FIG. 1a shows an illustration of one embodiment of the first aspect of the present invention. In FIG. 1a, the rod is in an undepressed position, i.e., prior to contacting the intraocular lens. The device 1 comprises a body 2 having a first end 3 and a second end 4. The body 2 has a lumen 5 which is located towards the first end 3 of the body 2. The lumen 5 has a cylindrical cross section. The body 2 has a load chamber 6 having a lumen 7, which when in the closed configuration is coaxial with the body lumen 5. However, in FIG. 1a, the load chamber 6 is shown in the open configuration. The load chamber 6 comprises a hinged plate 8 with a recess 9 having semi-circular cross-section which defines approximately one half of the lumen 7. The body 2 further comprises a second plate 10 (not shown) and a recess 11 (not shown) having semi-circular cross-section which defines the other half of the lumen 7. In use, the intraocular lens 12 may be loaded into the recess 11 of the load chamber 6 and the hinged plate 8 may be closed against the second plate 10. A tapered injector tip 13 is coupled to the first end 3 of the body 2 via a snap-fit lock 14. The injector tip 13 comprises a lumen having a tapering cross-sectional towards its open end 16. The open end 16 is cut at an angle relative to the longitudinal axis of the body lumen 5. This enables the open end of the tip 16 to be more easily pushed into a small incision in an eye.

A rod 17 having a main section 18 and a guide section 19 is located within the body lumen 5. The main section of the rod 18 has a first end 20 having a profiled, lens-contacting tip 21. The main section 18 has a truncated cylindrical cross-section having a recess 22 (not shown) running the length of its interface 23 with the guide section 19. The guide section 19 has a rail 24 (not shown) having a triangular cross-section which is complimentary to the recess 22. The second end 25 of the rod 17 has a circular plate 26 which the user contacts with their thumb or finger to depress the rod 17 into contact with the intraocular lens 12.

The guide section 19 has a first end 27 which is approximately co-terminus with the lens contacting tip 21. The guide section 19 has a second end 28 which has a hinge 28a and which is resiliently biased transverse to the longitudinal axis of the guide section 19.

In FIG. 1b the load chamber is shown in the closed configuration with the lens in situ. The first end of the rod 17 is located adjacent to the intraocular lens 12 in the load chamber 6.

In FIG. 1c. shows the device 1 in which the rod 17 has been depressed to the point at which its tip 21 comes into proximity with the start of the tapered inner wall of the injector tip. At this point the second end 28 of the guide section 19 comes into proximity with a recess 29 within the wall of the body lumen 5. The recess 29 is complimentary to the shape of the second end 28 and is capable of accommodating the second end 28 as it rotates about its hinge 28a. The second end 28 has a detente which engages a depression 31 in the surface of the main section 18 of the rod 17. This engagement prevents relative movement of the main section 18 relative to the guide section 19. However, in FIG. 1c, the détente 30 is shown having just come free of the depression 31.

FIG. 1d. shows the device 1 as the rod has been further depressed compared to the position shown in FIG. 1c. The second end 28 has been accommodated within the recess 29 and the détente 30 has become disengaged with the depression 31. The main section 18 of the rod 17 has been pushed further down the body lumen 5 and into the lumen 15 of the injector tip 13. The profiled, lens-contacting tip 21 is in contact with the intraocular lens 12 which has been pushed out of the opening 16 of the injector tip 13. It can be seen that the tip 21 has a largest diameter which is complimentary to the diameter of the inner surface of the tip lumen 15.

FIG. 2 shows an enlarged side view (not to scale) of the guide section 19, showing the first end 27. The guide section 19 has a second end 28 which has a hinge 28a and which is resiliently biased transverse to the longitudinal axis of the guide section 19. The second end 28 has a détente 30.

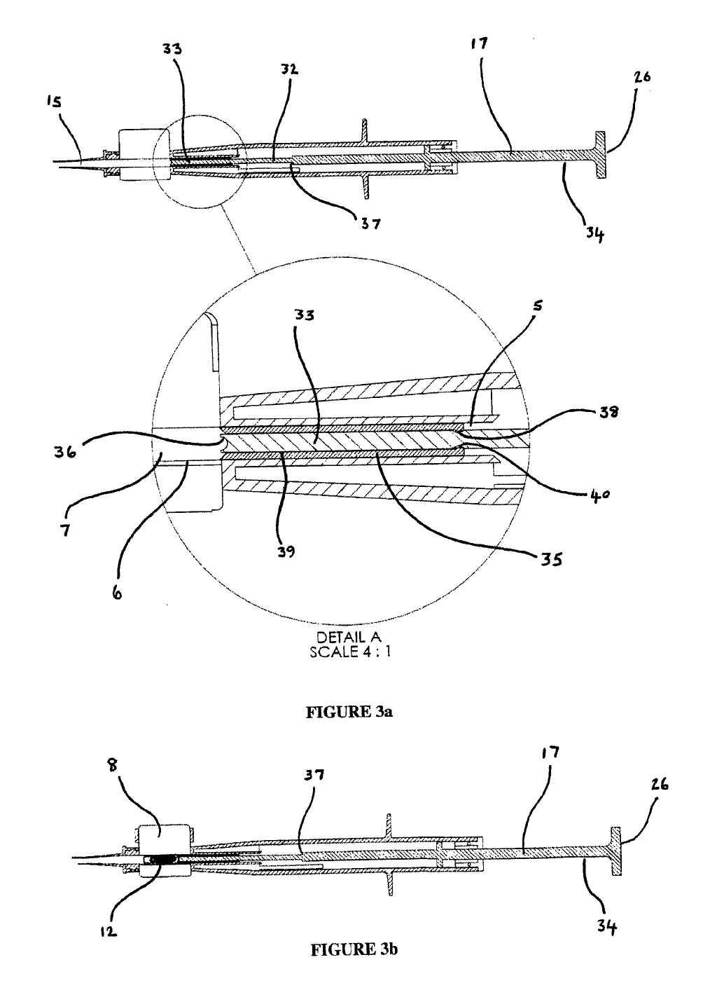

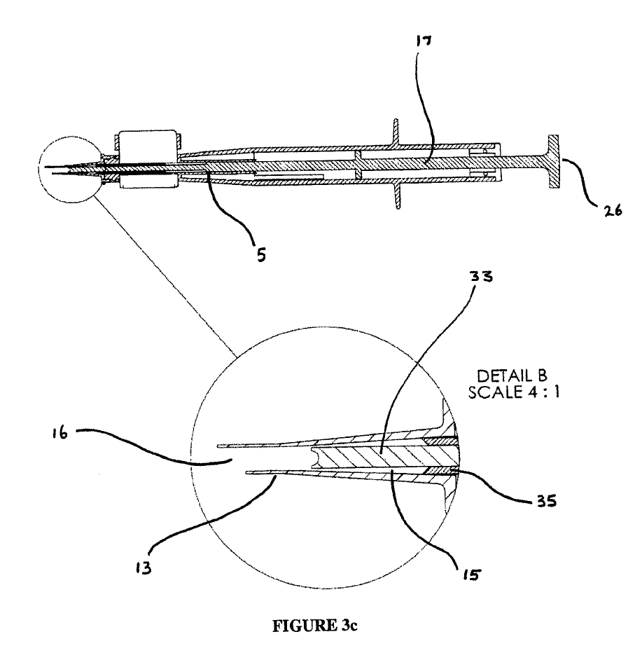

FIG. 3a shows a cross-sectional illustration of a second embodiment of a device according to the first aspect of the present invention. Furthermore, the FIG. 3a shows an exploded partial section of the body lumen section 5. The rod 17 comprises a main section 32 having a first end 33 and a second end 34. The second end has a circular plate 26 which the user contacts with their thumb or finger to depress the rod 17 into contact with the intraocular lens 12 (not shown). The first end 33 is cylindrical and has a smaller diameter than the second end 34. The first end comprises a sleeve 35 which is coaxial with the main section 32. The first end 33 has a lens-contacting tip 36. The sleeve is capable of sliding from the tip of the lens-contacting tip 36 to the junction 37 of the first end 33 and the second end 34. The sleeve 35 may be maintained in a first position by the presence of a détente 38 on inside surface 39 of the sleeve 35 which engages with a dimple 40 in the outer surface of the first end 33 of the rod 17. The dimple 40 is shallow and the détente 38 has a low profile, thereby allowing the détente 38 to disengage with the dimple 40 upon the application of a pre-determined pressure applied by depression of the rod 17 within the lumen 5.

FIG. 3b shows the, embodiment of FIG. 3a. but the rod 17 has now been advanced along the lumen 5 and into contact with the intraocular lens 12 within the lumen 7 of the load chamber 6.

FIG. 3c shows the embodiment of FIG. 3b. but the rod 17 has now been advanced along the lumen 5 and into the lumen 15 of the injector tip 13. Furthermore, the FIG. 3c shows an exploded partial section of the injector tip 13. The profiled, lens-contacting tip 36 is proximate the opening 16 of the injector tip 13, but the intraocular lens 12 is not shown. It can be seen that the first end 33 has a largest diameter which is complimentary to the diameter of the inner surface of the tip lumen 15. The first end 41 of the sleeve 35 has come into contact with the tapered surface of the inside of the lumen 15 of the injector tip 13. By virtue of the pressure applied to the rod 17 to move it along the lumen 5, the frictional resistance between the detente 38 and the dimple 40 has been overcome and the sleeve 35 remains in the same longitudinal position within the device as it cannot more into the restricted diameter of the tapering injector tip 13. The first end 33 of the rod has moved relative to the sleeve 35 and is in contact with the intraocular lens 12 that is proximate the opening 16 of the injector tip 13.

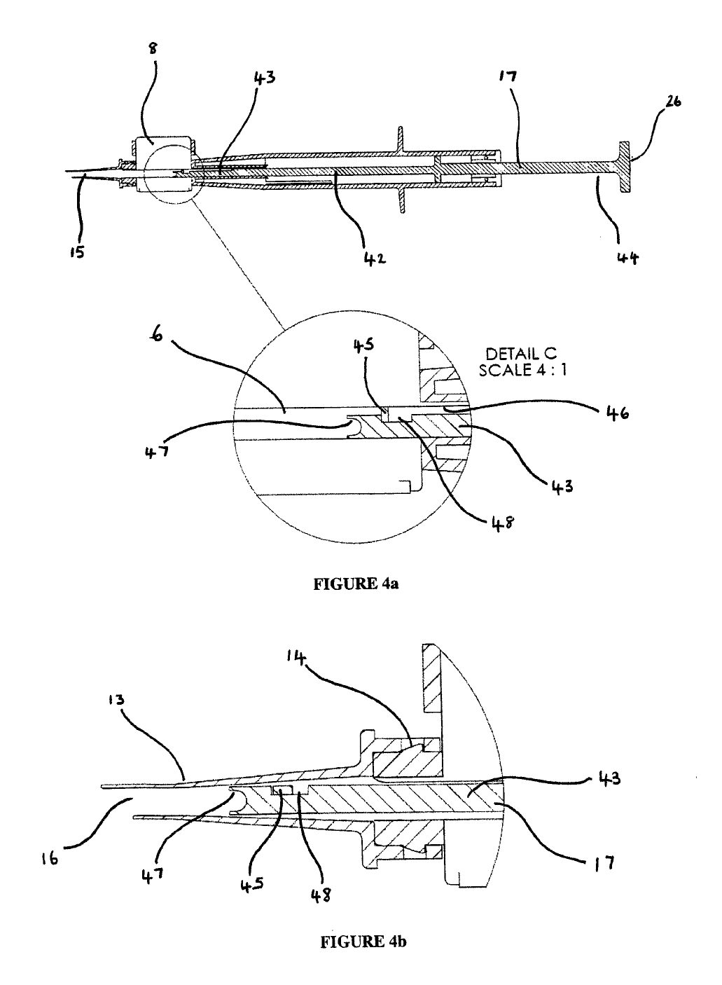

FIG. 4a shows a cross-sectional illustration of an embodiment of a device according to the second aspect of the present invention. Furthermore, the FIG. 4a shows an exploded partial section of the body lumen 5 and the load chamber 6. The rod 17 comprises a main section 42 having a first end 43 and a second end 44. The second end has a circular plate 26 which the user contacts with their thumb or finger to depress the rod 17 into contact with the intraocular lens 12. The first end 43 is cylindrical and has a smaller diameter than the second end 44. The first end comprises a projection 45 which extends to and engages a rail 46 which is located on the inner surface of the lumen 5. The first end 43 has a lens-contacting tip 47. The projection 45 has a weakened point of attachment with the first end 43. The first end has a recess 48 adjacent to the projection 45. The projection 45 has a depression 49 (not shown) which is complimentary to the profile of the rail 46 and allows the rod 17 to slide within the lumen 5 along the rail 46.

FIG. 4b shows a close up view of the injector tip 13 of the embodiment shown in FIG. 4a. but the rod 17 has now been advanced further along the lumen 5 and into the lumen 15 of the injector tip 13. The profiled, lens-contacting tip 47 is proximate the opening 16 of the injector tip 13. The first end 43 has a largest diameter which is complimentary to the diameter of the inner surface of the tip lumen 15. The projection 45 has come into contact with the tapered surface of the inside of the lumen 15 of the injector tip 13 which acts as a stop for the projection 45. By virtue of the pressure applied to the rod 17 to move it along the lumen 5, the projection 45 has bent at its weakened point of attachment with the first end 43 and has folded into the recess 48, thereby allowing the rod 17 to continue its movement towards the injector tip 13.

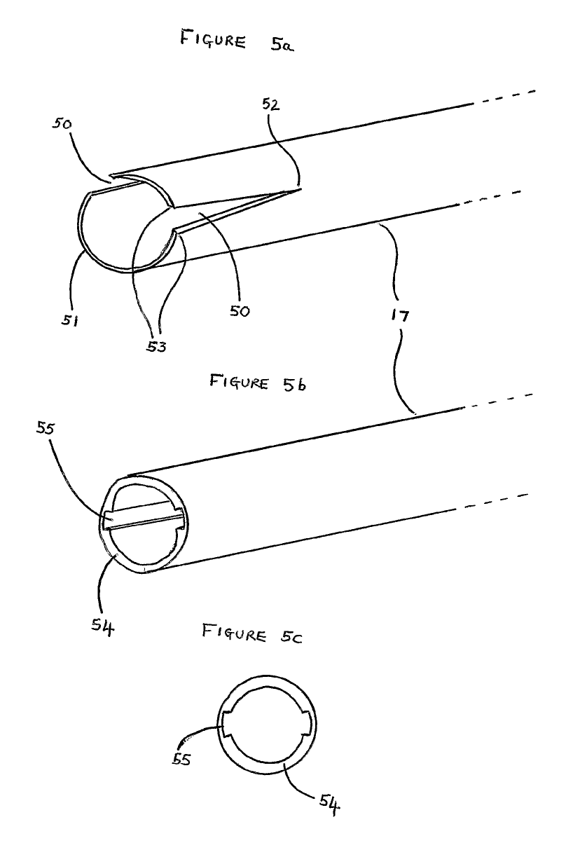

FIG. 5a shows a perspective view of the lens contacting tip portion of a rod according to the first aspect of the present invention. The rod 17 comprises two slots 50 cut into the wall 51 of the tubular rod 17. The slots taper away from the tip towards their terminus 52 which is a predetermined distance from the tip. This distance is preferably from 5 mm to 40 mm. In the non-collapsed configuration, the edges of the slot 53 are spaced apart, preferably by 0.1 mm to 3 mm. In the collapsed configuration, the edges of the slot 53 preferably abut one another. This preferably provides a conical or frustoconical shape to the end of the rod.

FIG. 5b shows a perspective view of the lens contacting tip portion of a rod according to the first aspect of the present invention. The rod 17 comprises a tubular structure having a wall 54 having two thinned portions 55 cut into the wall. These thinned portions shall be referred to as recessed rails. Under a predetermined radial pressure, these recessed rails collapse, preferably producing a conical or frustoconical shape to the end of the rod.

FIG. 5c shows an end view of the rod shown in FIG. 5b.

1. A device for inserting an intraocular lens into an eye, comprising a body having a lumen which is capable of accommodating an intraocular lens and a rod which is capable of pushing said intraocular lens out of the lumen and into the eye, wherein the rod has an intraocular lens-contacting tip which has a first configuration having a larger radial cross-sectional profile than a second configuration thereof, wherein, at a pre-determined point of longitudinal displacement towards the injector tip, at least a first part of the rod is capable of being selectively detached from one or more other parts of the rod thereby enabling relative longitudinal movement of the first part of the rod to the other part(s) of the rod.

2. A device according to claim 1, wherein there is a stepped transition between the first and second configurations of the rod tip.

3. A device according to claim 1, wherein the rod has only two tip configurations.

4. A device according to claim 1, wherein the cross-sectional profile of the second configuration of the rod tip is 5-95% smaller than the first configuration.

5. A device according to claim 1, wherein the rod comprises two parts which are adapted for longitudinal movement relative to one another.

6. A device according to claim 1, wherein the longitudinal position of the first rod part may be maintained relative to the other rod part(s) by the inclusion of restraining and/or attachment means.

7. A device according to claim 6, wherein the restraining and/or attachment means comprise a détente.

8. A device according to claim 1, wherein the rod may comprise at least two parts having a telescoping action.

9. A device according to claim 8, wherein the rod comprises a main, substantially cylindrical portion having one or more co-axial sleeves or collars.

10. A method of delivering an intraocular lens to an eye, comprising placing an intraocular lens into a device according to claim 1, inserting the rod into the proximal end of the body lumen thereof, inserting the open mouth of an injector tip of the device through an incision in the eye, and urging the intraocular lens from the device through the injector tip and into the eye.
